(19) United States
(12) Patent Application Publication
(10) Pub. No.: US 2014/0187918 A1
(43) Pub. Date: Jul. 3, 2014

(54) POSITION DETECTION DEVICE, CAPSULE ENDOSCOPE SYSTEM, AND COMPUTER READABLE RECORDING MEDIUM

(71) Applicant: OLYMPUS MEDICAL SYSTEMS CORP., Tokyo (JP)

(72) Inventors: Naoya HIGAKI, Tokyo (JP); Masatoshi HOMAN, Tokyo (JP); Jun HASEGAWA, Tokyo (JP)

(73) Assignee: OLYMPUS MEDICAL SYSTEMS CORP., Tokyo (JP)

(21) Appl. No.: 14/098,750

(22) Filed: Dec. 6, 2013

Related U.S. Application Data
(63) Continuation of application No. PCT/JP2013/062505, filed on Apr. 26, 2013.

(30) Foreign Application Priority Data
Apr. 26, 2012 (JP) 2012-101447

(51) Int. Cl. A61B 1/04; A61B 1/00; A61B 5/06

(52) U.S. Cl. CPC A61B 1/041; A61B 5/061; A61B 1/00016; A61B 1/00045
USPC 600/424

(57) ABSTRACT

A position detection device includes: a region acquiring unit that acquires a region where at least two of spheres with receiving antennas as centers and distances between each receiving antenna and a capsule endoscope as radii overlap with one another; an orientation estimating unit that estimates orientations of the capsule endoscope at points in the region based on positional relationships between the points and the receiving antennas, and received strength of signals received by the receiving antennas; and a position determination unit that determines the position of the capsule endoscope based on the positions of the points and the orientations, wherein the position determination unit calculates theoretical values of the received strength at the points, acquires measured values of the received strength at the receiving antennas, and detects a point having a minimum error between the theoretical value and the measured value as the position of the capsule endoscope.

### CROSS REFERENCES TO RELATED APPLICATIONS

This application is a continuation of PCT international application Ser. No. PCT/JP2013/062505 filed on Apr. 26, 2013 which designates the United States, incorporated herein by reference, and which claims the benefit of priority from Japanese Patent Applications No. 2012-101447, filed on Apr. 26, 2012, incorporated herein by reference.

### BACKGROUND OF THE INVENTION

1. Field of the Invention

The present invention relates to a position detection device that detects a position of a capsule endoscope in a subject, a capsule endoscope system including the position detection device, and a computer readable recording medium.

2. Description of the Related Art

In the endoscopic field, a capsule endoscope containing an imaging function, a wireless communication function, and the like in a capsule-shaped casing formed in size insertable into the digestive tract of a subject such as a patient is conventionally known. The capsule endoscope is swallowed from the mouth of the subject and then moves in the subject such as in the digestive tract by the peristaltic movement and the like. Images of the inside of the subject are sequentially captured to generate image data. The image data are sequentially transmitted wirelessly.

The image data transmitted wirelessly from the capsule endoscope are received by a receiving device provided to the outside of the subject, and stored in a memory embedded in the receiving device. After the end of the examination, the image data accumulated in the memory of the receiving device are captured in an image display device. An observer such as a doctor observes an organ image and the like that are displayed by the image display device, and diagnoses the subject.

The capsule endoscope moves in the body cavity by the peristaltic movement and the like and accordingly it is required to correctly identify where in the body cavity the image data transmitted wirelessly from the capsule endoscope are captured. Hence, known is a medical system and the like that receive electromagnetic waves transmitted by the capsule endoscope by a plurality of receiving antennas provided outside the body cavity, and estimate the position of the capsule endoscope with the received strength of a plurality of received wireless signals, and the like (for example, see Japanese Laid-open Patent Publication No. 2008-99734, Japanese Laid-open Patent Publication No. 2006-68501 and Japanese Laid-open Patent Publication No. 2007-283001).

For example, Japanese Laid-open Patent Publication No. 2008-99734 discloses a technology for estimating the position of a capsule endoscope based on the received strength of an antenna, estimating travel speed and travel direction of the capsule endoscope in predetermined situations such as where reception is poor, and estimating the position of the capsule endoscope based on these travel speed, travel direction, and received strength.

Moreover, Japanese Laid-open Patent Publication No. 2006-68501 discloses a magnetic guidance medical system that controls the position and posture of a capsule-shaped medical device in the body by magnetism. In the magnetic guidance medical system, third-angle projection is applied to calculate a three-dimensional position of the capsule-shaped medical device based on the received strength of a wireless signal transmitted from the capsule-shaped medical device.

Moreover, Japanese Laid-open Patent Publication No. 2007-283001 discloses a capsule-shaped medical device that estimates the position of a capsule endoscope in the body and calculates the locus. In the capsule-shaped medical device, the estimation of the position and orientation of an antenna contained in a capsule endoscope is repeated by the Gauss-Newton method.

### SUMMARY OF THE INVENTION

According to one aspect of the present invention, a position detection device for detecting a position of a capsule endoscope in a subject, the capsule endoscope being introduced into the subject and moving in the subject, based on received strength of signals transmitted from the capsule endoscope at a plurality of receiving antennas, includes: a region acquiring unit that obtains distances between the receiving antennas and the capsule endoscope based on the received strength of the signals received by the plurality of receiving antennas, and acquires a region where at least two spheres of a plurality of spheres with the receiving antennas as centers and the distances corresponding respectively to the receiving antennas as radii overlap with one another; an orientation estimating unit that estimates orientations of the capsule endoscope at a plurality of points in the region based on positional relationships between the points and the receiving antennas, and the received strength of the signals received by the receiving antennas; and a position determination unit that determines the position of the capsule endoscope in the subject based on the positions of the points and the orientations of the capsule endoscope, wherein the position determination unit calculates theoretical values of the received strength of the signals received by the plurality of receiving antennas based on the positions of the points and the orientations of the capsule endoscope in a case of assuming that the capsule endoscope exists at the points, as well as acquiring measured values of the received strength at the plurality of receiving antennas, and detects a point having a minimum error between the theoretical value and the measured value as the position of the capsule endoscope out of the plurality of points.

According to another aspect of the present invention, a capsule endoscope system includes: a capsule endoscope that is introduced into a subject and moves in the subject to acquire image information inside the subject; and a position detection device that detects a position of the capsule endoscope in the subject based on received strength of signals transmitted from the capsule endoscope at a plurality of receiving antennas, the position detection device including a region acquiring unit for obtaining distances between the receiving antennas and the capsule endoscope based on the received strength of the signals received by the plurality of receiving antennas, and acquiring a region where at least two spheres of a plurality of spheres with the receiving antennas as centers and the distances corresponding respectively to the receiving antennas as radii overlap with one another, an orientation estimating unit for estimating orientations of the capsule endoscope at a plurality of points in the region based on positional relationships between the points and the receiving antennas, and the received strength of the signals received at the receiving antennas, and a position determination unit for determining the position of the capsule endoscope in the subject based on the positions of the points and the orientations of the capsule endoscope, wherein the position determination unit calculates theoretical values of the received strength of the signals received by the plurality of receiving antennas based on the positions of the points and the orientations of the capsule endoscope in a case of assuming that the capsule endoscope exists at the points, as well as acquiring measured values of the received strength at the plurality of receiving antennas, and detects a point having a minimum error between the theoretical value and the measured value as the position of the capsule endoscope out of the plurality of points.

According to still another aspect of the present invention, a capsule endoscope system includes: a capsule endoscope that is introduced into a subject and moves in the subject to acquire image information inside the subject; a receiving device including a plurality of antennas for receiving a signal including the image information transmitted from the capsule endoscope, a region acquiring unit for obtaining distances between the receiving antennas and the capsule endoscope based on received strength of the signals received by the plurality of receiving antennas, and acquiring a region where at least two spheres of a plurality of spheres with the receiving antennas as centers and the distances corresponding respectively to the receiving antennas as radii overlap with one another, an orientation estimating unit for estimating orientations of the capsule endoscope at a plurality of points in the region based on positional relationships between the points and the receiving antennas, and the received strength of the signals received by the receiving antennas, and a position determination unit for determining a position of the capsule endoscope in the subject based on the positions of the points and the orientations of the endoscope; and an image display unit that acquires the image information and position information indicating the position of the capsule endoscope corresponding to the image information from the receiving device and displays an image corresponding to the image information and the position of the capsule endoscope, wherein the position determination unit calculates theoretical values of the received strength of the signals received by the plurality of receiving antennas based on the positions of the points and the orientations of the capsule endoscope in a case of assuming that the capsule endoscope exists at the points, as well as acquiring measured values of the received strength at the plurality of receiving antennas, and detects a point having a minimum error between the theoretical value and the measured value as the position of the capsule endoscope out of the plurality of points.

According to yet another aspect of the present invention, a non-transitory computer readable recording medium where an executable program is stored, the program instructing a processor to execute the following: obtaining distances between receiving antennas and a capsule endoscope that is introduced into a subject and moves in the subject, based on received strength at the receiving antennas upon the receiving antennas receiving signals transmitted from the capsule endoscope, and acquiring a region where at least two spheres of a plurality of spheres with the receiving antennas as centers and the distances corresponding respectively to the receiving antennas as radii overlap with one another; estimating orientations of the capsule endoscope at a plurality of points in the region based on positional relationships between the points and the receiving antennas, and the received strength of the signals received by the receiving antennas; determining a position of the capsule endoscope in the subject based on the positions of the points and the orientations of the capsule endoscope; and upon determination of the position, calculating theoretical values of the received strength of the signals received by the plurality of receiving antennas based on the positions of the points and the orientations of the capsule endoscope in a case of assuming that the capsule endoscope exists at the points, as well as acquiring measured values of the received strength at the plurality of receiving antennas, and detecting a point having a minimum error between the theoretical value and the measured value as the position of the capsule endoscope out of the plurality of points.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic diagram illustrating a configuration of a capsule endoscope system according to a first embodiment of the present invention;

FIG. 2 is a schematic diagram illustrating an internal configuration of a capsule endoscope illustrated in FIG. 1;

FIG. 3 is a block diagram illustrating a schematic configuration of an information processing device illustrated in FIG. 1;

FIG. 4 is a flowchart illustrating the operation of the information processing device illustrated in FIG. 1;

FIG. 5 is a flowchart illustrating the details of a position determination process illustrated in FIG. 4;

FIG. 6 is a schematic diagram illustrating a process of acquiring an existing region;

FIG. 7 is an enlarged view of the existing region illustrated in FIG. 6;

FIG. 8 is a flowchart illustrating the details of a process of estimating the orientation of the capsule endoscope;

FIG. 9 is a schematic diagram illustrating the process of estimating the orientation of the capsule endoscope;

FIG. 10 is a schematic diagram illustrating the process of estimating the orientation of the capsule endoscope;

FIG. 11 is a diagram illustrating a calculation principle of a theoretical value of received strength at each receiving antenna;

FIG. 12 is a diagram illustrating the calculation principle of the theoretical value of the received strength at each receiving antenna;

FIG. 13 is a diagram illustrating the calculation principle of the theoretical value of the received strength at each receiving antenna;

FIG. 14 is a diagram illustrating the calculation principle of the theoretical value of the received strength at each receiving antenna;

FIG. 15 is a diagram illustrating a process of correcting the moving locus of the capsule endoscope;

FIG. 16 is a diagram illustrating the process of correcting the moving locus of the capsule endoscope;

FIG. 17 is a schematic diagram illustrating another method for acquiring the existing region of the capsule endoscope;

FIG. 18 is a schematic diagram illustrating another configuration example of the capsule endoscope system according to the first embodiment of the present invention; and

FIG. 19 is a block diagram illustrating a configuration example of a receiving device included in a capsule endoscope system according to a second embodiment of the present invention.

### DETAILED DESCRIPTION OF THE PREFERRED EMBODIMENTS

Hereinafter, a description will be given of a position detection device, a capsule endoscope system, and a computer readable recording medium according to embodiments of the present invention with reference to the drawings. In the following description, a capsule endoscope system including a capsule endoscope that is inserted into the body of a subject and captures an in-vivo image of the subject is illustrated as an example of the position detection device and the capsule endoscope system according to the present invention. However, the embodiments shall not limit the present invention.

### First Embodiment

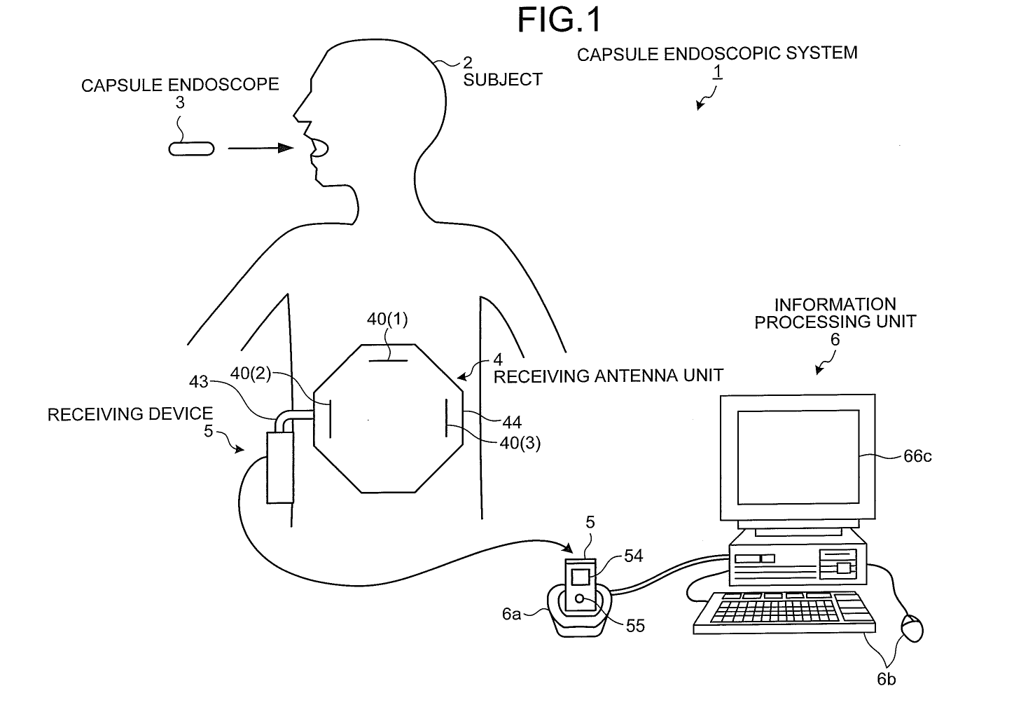

FIG. 1 is a schematic diagram illustrating a configuration of a capsule endoscope system 1 according to a first embodiment of the present invention. As illustrated in FIG. 1, the capsule endoscope system 1 includes a capsule endoscope 3 that captures an in-vivo image of a subject 2, a receiving device 5 that receives via a receiving antenna unit 4 a wireless signal transmitted wirelessly from the capsule endoscope 3 introduced into the subject 2, and an information processing device 6 that estimates an imaging position of an image obtained by capturing the inside of the subject 2 by the capsule endoscope 3 and displays an image of the inside of the subject 2 based on the image data obtained by being captured by the capsule endoscope 3.

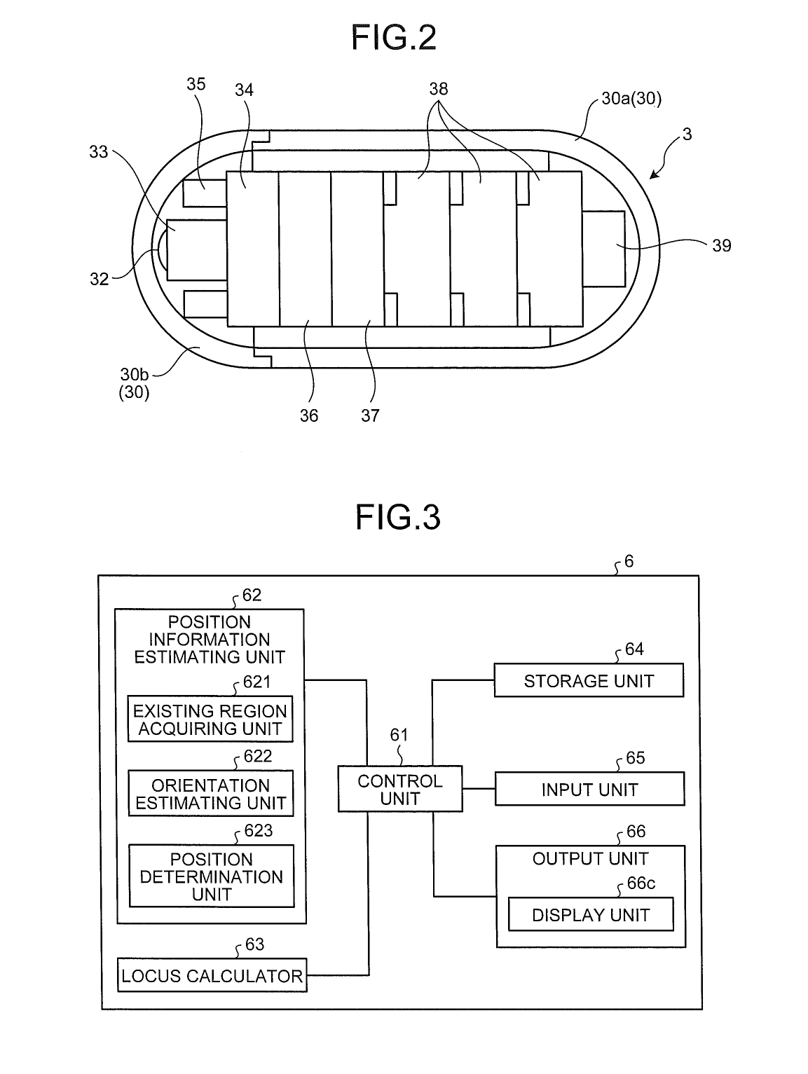

FIG. 2 is a schematic diagram illustrating an internal configuration of the capsule endoscope 3. As illustrated in FIG. 2, the capsule endoscope 3 is housed in a capsule-shaped container 30 (casing) including a substantially cylindrical shaped or a semi-elliptical shaped container 30a forming a hemispherical dome at one end, and opening at the other end, and a hemispherical optical dome 30b that is inserted into an opening of the container 30a to make the container 30a watertight. The capsule-shaped container 30 (30a and 30b) is, for example, the size to the degree that the subject 2 can swallow. Moreover, in the first embodiment, at least the optical dome 30b is formed with transparent material.

The capsule endoscope 3 includes an objective lens 32 that forms an image with light incident through the optical dome 30b, a lens frame 33 to which the objective lens 32 is attached, an imaging unit 34 that converts an optical signal incident from the objective lens 32 into an electrical signal and forms an imaging signal, a lighting unit 35 that illuminates the inside of the subject 2 upon image capture, a circuit board 36 where a processing circuit that drives the imaging unit 34 and the lighting unit 35 and generates an image signal from the imaging signal input from the imaging unit 34, and the like are formed, a transmitting/receiving circuit 37 that transmits the image signal and receives signals from the receiving device 5 and the like outside the body cavity, a plurality of button cells 38 that supplies power to these function units, and an antenna 39 in, for example, a circular coil form or circular loop form.

After being swallowed by the subject 2, the capsule endoscope 3 passes through the esophagus in the subject 2 and moves in the body cavity by the peristaltic movement of the lumen of the digestive tract. The capsule endoscope 3 consecutively captures the inside of the body cavity of the subject 2 at minute time intervals, for example, at 0.5-second intervals while moving in the body cavity, and generates image data to sequentially transmit the image data to the receiving device 5. In the first embodiment, it is also possible to execute a position estimation process by an image signal of image data generated by the imaging unit 34 of the capsule endoscope 3 capturing an image. However, it is more preferred to generate a transmission signal including the generated image signal and a received strength detection signal for detecting the position of the capsule endoscope 3 and execute a position detection process by the received strength detection signal that is easy to detect received strength.

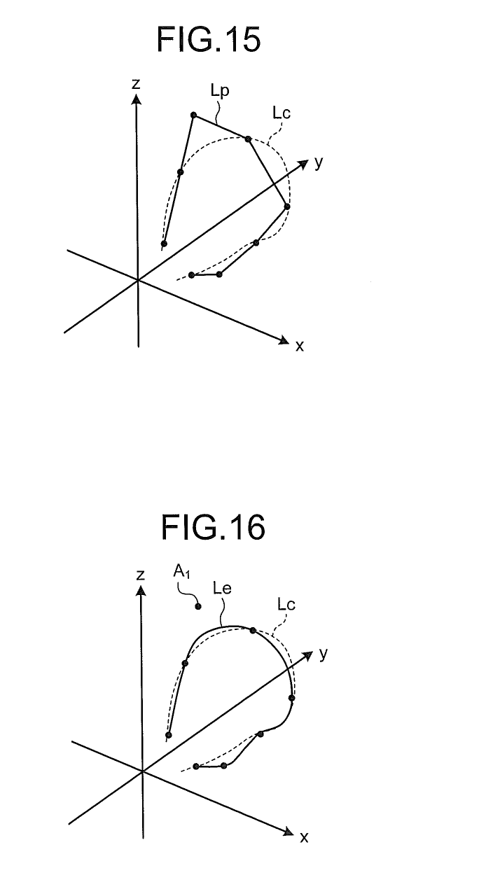

The receiving device 5 is connected by the sheet-shaped receiving antenna unit 4 where a plurality of (three in FIG. 1) receiving antennas 40(1) to 40(3) are arranged, and an antenna cable 43. The receiving device 5 receives wireless signals transmitted from the capsule endoscope 3 respectively via the receiving antennas 40(1) to 40(3) and detects the received field strength of the wireless signal for each of the receiving antennas 40(1) to 40(3) as well as acquiring image data of the inside of the subject 2 based on the received wireless signals. The receiving device 5 associates received field strength information of the receiving antennas 40(1) to 40(3), time information indicating a time, and the like with the received image data and stores the image data in a storage unit (see FIG. 15) to be described below. Moreover, the receiving device 5 may include a display unit 54 that displays an image corresponding to image data received from the capsule endoscope 3, and an operating unit 55 used when an instruction operation on the receiving device 5 is input.

It is preferred that the receiving antennas 40(1) to 40(3) be antennas using only main polarization (in other words, not using cross polarization). In the embodiment, dipole antennas are used as the receiving antennas 40(1) to 40(3). Hereinafter, these receiving antennas 40(1) to 40(3) are also described as a receiving antenna 40(n) (n=1 to N, N=3 in FIG. 1).

The receiving device 5 is carried by the subject 2 while the capsule endoscope 3 is capturing images, in other words, during a period of time from when the capsule endoscope 3 is inserted, for example, from the mouth of the subject 2 to when it passes through the digestive tract and is discharged from the subject 2. The receiving device 5 is removed from the subject 2 after the end of the examination with the capsule endoscope 3, and is connected to the information processing device 6 to transfer information on the image data received from the capsule endoscope 3, and the like.

The receiving antennas 40(n) are arranged in predetermined positions of a sheet 44, for example, positions corresponding to the organs in the subject 2, the organs being a passage route of the capsule endoscope 3, when the receiving antenna unit 4 is attached to the subject 2. The arrangement of the receiving antennas 40(n) can be freely changed according to the purpose such as an examination or diagnosis.

The information processing device 6 is configured using a work station or a personal computer including a display unit 66c of a liquid crystal display or the like. The information processing device 6 is an image display device that displays an image corresponding to image data of the inside of the subject 2 acquired via the receiving device 5 and is also a position detection device that detects the position of the capsule endoscope 3 when the image was captured.

The information processing device 6 is connected to a cradle 6a that reads image data and the like from the storage unit of the receiving device 5 and an operation input device 6b such as a keyboard and a mouse. The cradle 6a acquires image data and associated information associated with the image data, such as received field strength information, time information, and identification information of the capsule endoscope 3, from a memory of the receiving device 5 when the receiving device 5 is worn, and transfers the acquired various pieces of information to the information processing device 6. The operation input device 6b accepts input by a user. Consequently, the user observes living body parts in the subject 2, for example, esophagus, stomach, small intestine, and large intestine while operating the operation input device 6b and watching images of the inside of the subject 2 that are sequentially displayed by the information processing device 6, and diagnoses the subject 2.

Next, a configuration of the information processing device 6 illustrated in FIG. 1 will be described in detail. FIG. 3 is a block diagram illustrating the configuration of the information processing device 6 illustrated in FIG. 1. As illustrated in FIG. 3, the information processing device 6 includes a control unit 61 that performs centralized control of the entire information processing device 6, a position information estimating unit 62 that estimates the position of the capsule endoscope 3 at a timing when a wireless signal including image data is received, and generates position information, a locus calculator 63 that calculates the moving locus of the capsule endoscope 3 in the subject 2 based on the position information of the capsule endoscope 3 generated by the position information estimating unit 62 on the basis of each image data, a storage unit 64 that stores image data received from the capsule endoscope 3 and signal strength, an input unit 65 that acquires information from the operation input device 6b such as a keyboard and a mouse, and the like, and an output unit 66 that includes the display unit 66c configured by a display and is configured using a printer, a speaker, and the like. The storage unit 64 is configured using a hard disk that magnetically stores information, and a memory that loads from the hard disk various programs, parameters, and the like that are related to a process when the capsule endoscope system 1 executes the process, and electrically stores them.

The position information estimating unit 62 acquires the strength of signals respectively received by the plurality of receiving antennas 40(n) of the receiving antenna unit 4 (the received field strength, hereinafter also simply referred to as the received strength), and estimates the position of the capsule endoscope 3 (the position of the built-in antenna 39) from the strength of these signals. The position information estimating unit 62 includes an existing region acquiring unit 621, an orientation estimating unit 622, and a position determination unit 623.

The existing region acquiring unit 621 estimates the existing region of the capsule endoscope 3 at a position detection timing based on the received strength at the receiving antennas 40(n).

The orientation estimating unit 622 estimates the orientation of the capsule endoscope 3, in other words, the orientation of the antenna 39 contained in the capsule endoscope 3 based on positional relationships between a plurality of points in the existing region and the receiving antennas 40 (n), and the received strength at the receiving antennas 40(n) when the capsule endoscope 3 is assumed to exist at each of the plurality of points.

The position determination unit 623 detects a more detailed position of the capsule endoscope 3 in the existing region based on the positions of the points in the existing region, and the orientations of the capsule endoscope 3 estimated by the orientation estimating unit 622.

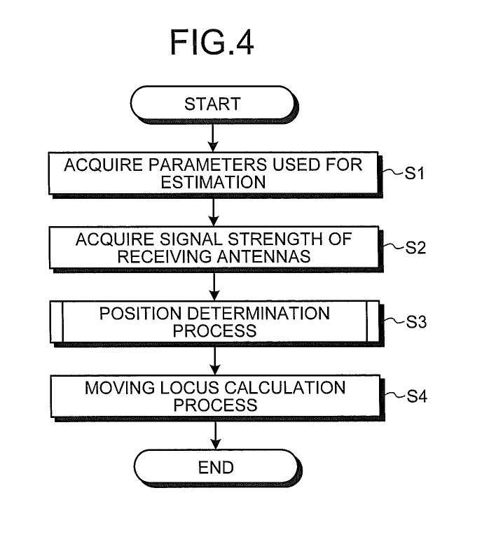

Next, the operation of the information processing device 6 illustrated in FIG. 1 will be described. FIG. 4 is a flowchart illustrating the operation of the information processing device 6.

Firstly, in Step S1, the position information estimating unit 62 acquires from the storage unit 64 parameters used to estimate the position of the capsule endoscope 3. The content of the parameters is described below.

In the following Step S2, the information processing device 6 acquires the signal strength of a signal received by each receiving antenna 40(n) at the position detection timing.

In the following Step S3, the position information estimating unit 62 determines the position of the capsule endoscope 3 in the subject 2 based on the received strength at each receiving antenna 40(n), and creates position information.

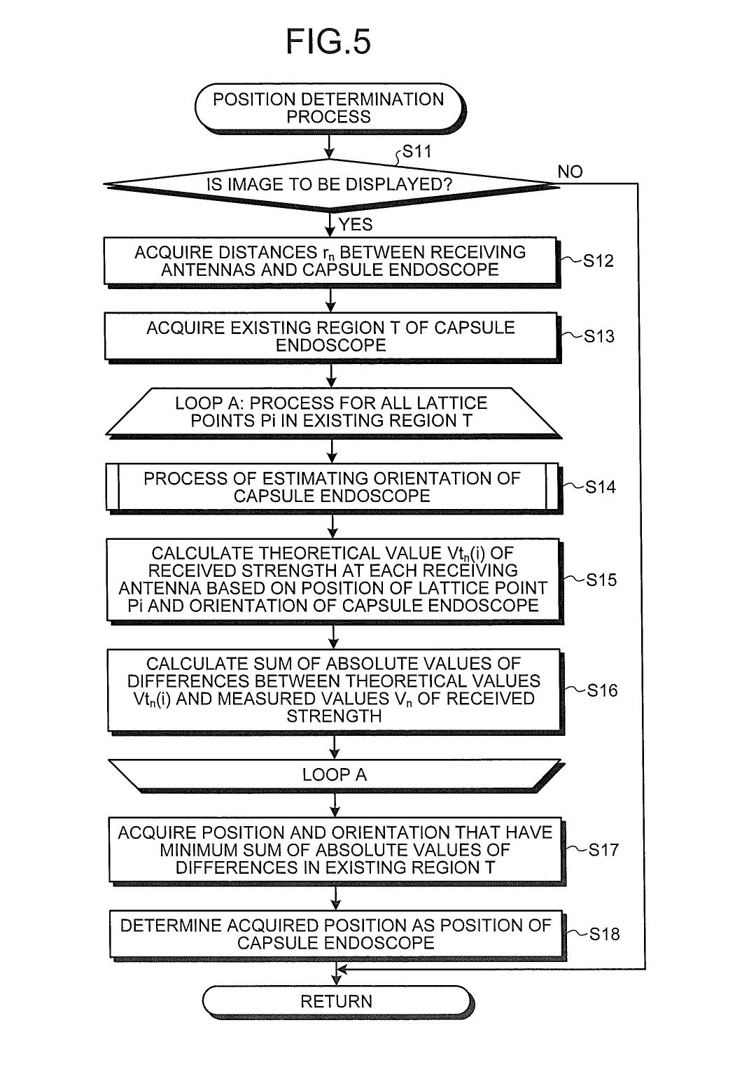

FIG. 5 is a flowchart illustrating the details of a position determination process executed by the position information estimating unit 62.

Firstly, in Step S11, the position information estimating unit 62 judges whether or not an image corresponding to image data input into the information processing device 6 is an image to be displayed on the display unit 66c. For example, if the ratio of noise included in image data is larger than a predetermined threshold value, it is judged that an image corresponding to the image data is not an image to be displayed on the display unit 66c. If an image corresponding to image data input is not an image to be displayed on the display unit 66c (Step S11: No), the processing returns to the main routine. This is because such an image is judged to be an image unsuitable for the user to observe.

If an image corresponding to image data input is an image to be displayed on the display unit 66c (Step S11: Yes), the existing region acquiring unit 621 acquires distances rₙ between the receiving antennas 40(n) and the capsule endoscope 3 from the received strength at the receiving antennas 40n (Step S12).

In more detail, the existing region acquiring unit 621 obtains voltage Vₙ of the received signal based on the received field strength at the receiving antenna 40(n), and calculates the distance rₙ using the following equation (1).

$${V}_{n} = K \frac{1}{{r}_{n}} {e}^{- α {r}_{n}} \qquad ( 1 )$$

In equation (1), the parameter K is a constant determined by the characteristic of the receiving antenna 40(n), and the parameter α is an attenuation coefficient of biological tissue. These parameters K and α are derived from actual measured values previously measured, and acquired from the storage unit 64 in Step S1.

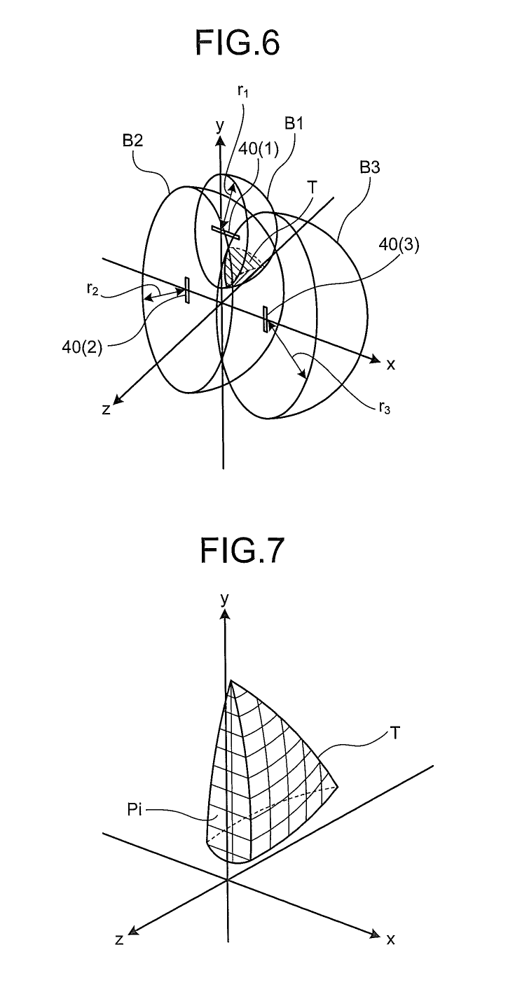

In the following Step S13, the existing region acquiring unit 621 acquires a region where the capsule endoscope 3 exists (existing region) T based on the distances rₙ between the receiving antennas 40(n) and the capsule endoscope 3. In more detail, a region where at least two or more spheres of a plurality of spheres with the receiving antennas 40(n) as centers and the distances rₙ as radii overlap with one another is acquired as the existing region T. For example, in a case of the receiving antennas 40(1) to 40(3), as illustrated in FIG. 6, a region where a sphere B1 with the receiving antenna 40(1) as a center and a distance r₁ as a radius, a sphere B2 with the receiving antenna 40(2) as a center and a distance r₂ as a radius, and a sphere B3 with the receiving antenna 40(3) as a center and a distance r₃ as a radius overlap with one another is acquired as the existing region T of the capsule endoscope 3. If the coordinates of the receiving antennas 40(1) to 40(3) are taken on such a X-Y plane as illustrated in FIG. 6, the spheres B1 to B3 where the capsule endoscope 3 is estimated to be located can be considered to be, for example, hemispheres on the Z≦0 side (the subject 2 side).

FIG. 7 is an enlarged view of the existing region T illustrated in FIG. 6. Following Step S13, the position information estimating unit 62 executes a process of a loop A on points (for example, center points. Hereinafter referred to as the lattice points) Pi in a plurality of sub-regions obtained by dividing the existing region T in a lattice form.

In Step S14, the orientation estimating unit 622 executes a process of estimating the orientation of the capsule endoscope 3 when the capsule endoscope 3 is assumed to exist at the lattice point Pi.

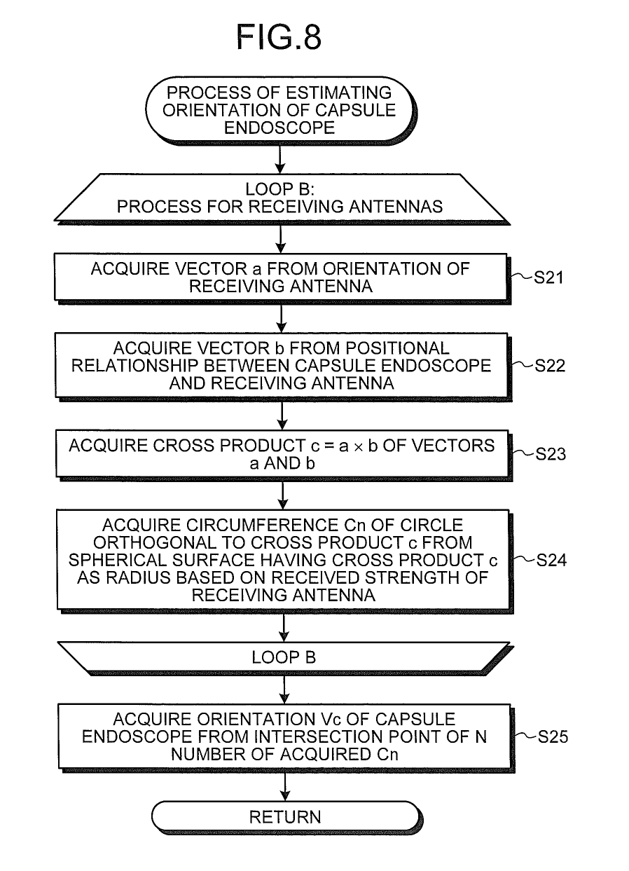

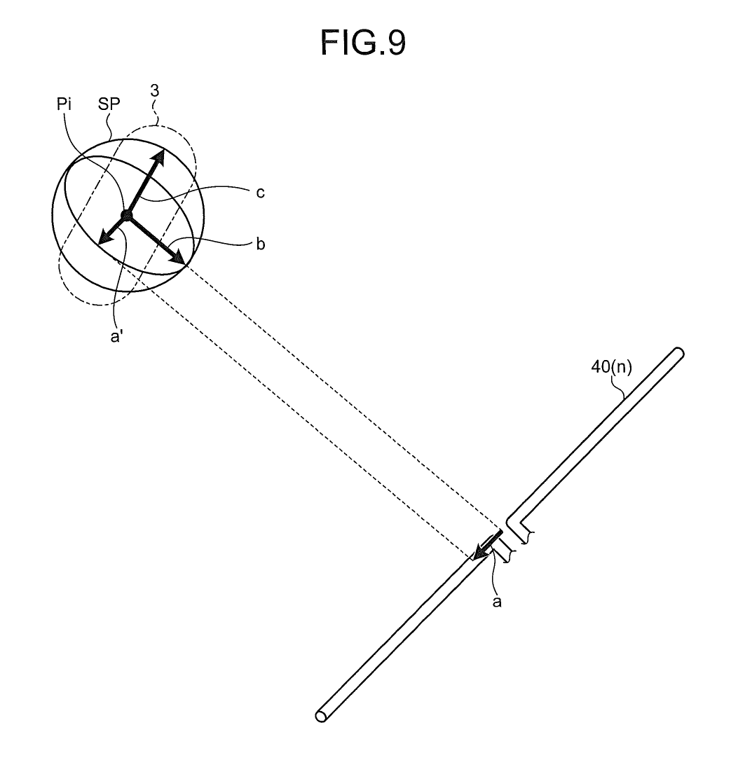

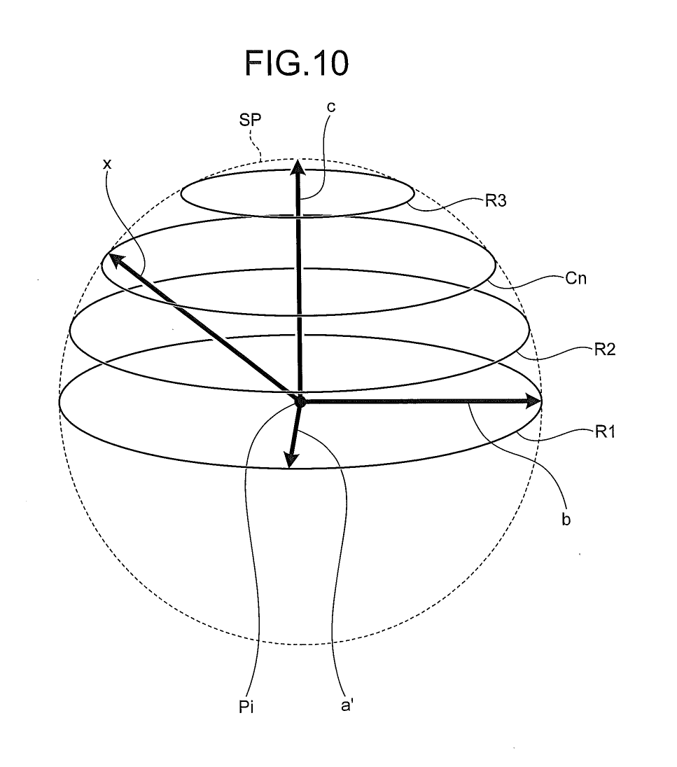

FIG. 8 is a flowchart illustrating the details of the process of estimating the orientation of the capsule endoscope 3, the process being executed by the orientation estimating unit 622. Moreover, FIGS. 9 and 10 are schematic diagrams illustrating the process of estimating the orientation of the capsule endoscope 3.

As illustrated in FIG. 8, the orientation estimating unit 622 executes a process of a loop B on each of the receiving antennas 40(n) (n=1 to N, n is a natural number).

Firstly, in Step S21, the orientation estimating unit 622 acquires a vector a that represents the orientation of the receiving antenna 40(n) being a process target as illustrated in FIG. 9. FIG. 9 also illustrates, as a vector a′, a vector that is the vector a moved parallel to the center (in other words, the lattice point Pi) of the capsule endoscope 3.

In the following Step S22, the orientation estimating unit 622 acquires a vector b pointing to the receiving antenna 40(n) from the capsule endoscope 3 (the lattice point Pi) based on the positional relationship between the capsule endoscope 3 and the receiving antenna 40(n).

In the following Step S23, the orientation estimating unit 622 acquires a vector c being the cross product of the vector a (in other words, the vector a′) and the vector b (c=a×b).

Furthermore, in Step S24, the orientation estimating unit 622 acquires a circumference Cn of a circle orthogonal to the vector c based on a spherical surface SP having the vector c as a radius based on the received strength at the receiving antenna 40(n). As illustrated in FIG. 10, the orientation of the capsule endoscope 3 (in other words, the orientation of the antenna 39 contained in the capsule endoscope 3) can be expressed as a vector starting from the same starting point (the lattice point Pi) as the vector c and having any point on the spherical surface SP as an endpoint (hereinafter referred to as a vector x). At this point, in terms of the circumference of the circle orthogonal to the vector c (for example, circumferences R1, R2, . . . , and so forth), even if the vector x passes at any point on one circumference, a theoretical value of the received strength at the antenna 40(n) does not change. Hence, the orientation estimating unit 622 acquires one circumference Cn orthogonal to the vector c as a set of endpoint candidates of the vector x based on the received strength of the antenna 40(n).

After the circumferences Cn are acquired for all (N number) of the receiving antennas 40(n), in Step S25, the orientation estimating unit 622 obtains an intersection point of N number of the circumferences Cn, and acquires an orientation Vc of the capsule endoscope 3 (the antenna 39) from the intersection point. Afterwards, the processing returns to the main routine.

In Step S15 (see FIG. 5) following Step S14, the position determination unit 623 calculates a theoretical value Vtₙ(i) of the received strength at each of the receiving antennas 40(n) from the position of the lattice point Pi and the orientation Vc of the capsule endoscope 3.

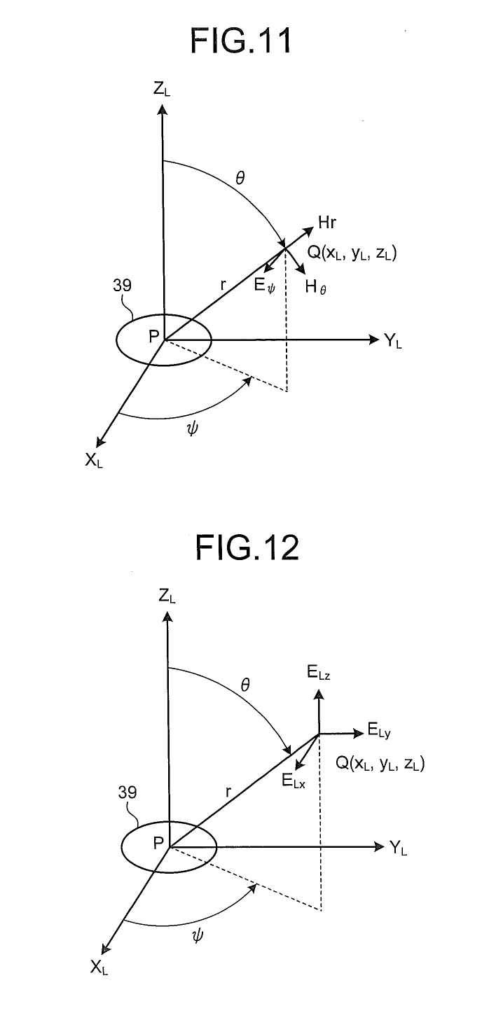

As illustrated in FIG. 11, in a coordinate system X_{L}Y_{L}Z_{L} (an origin P) relative to the circular coil or circular loop shaped antenna 39 placed in the capsule endoscope 3, magnetic-field components Hᵣ and H_{θ} of a magnetic field (including components of an electrostatic field, a radiation field, and an induction filed), and an electric field component E_{ψ} at an arbitrary point Q (x_{L}, y_{L}, z_{L}) are expressed by the following equations (2-1) to (2-3).

$$\begin{matrix}{H}_{r} = \frac{IS}{2 π} ( \frac{j k}{{r}^{2}} + \frac{1}{{r}^{3}} ) \cdot {e}^{- j kr} \cdot cos θ & ( 2 \text{-} 1 ) \\ {H}_{θ} = \frac{IS}{4 π} ( - \frac{{k}^{2}}{r} + \frac{j k}{{r}^{2}} + \frac{1}{{r}^{3}} ) \cdot {e}^{- j kr} \cdot sin θ & ( 2 \text{-} 2 ) \\ {E}_{ψ} = - \frac{j ω μ IS}{4 π} ( \frac{j k}{r} + \frac{1}{{r}^{2}} ) \cdot {e}^{- j kr} \cdot sin θ & ( 2 \text{-} 3 )\end{matrix}$$

In equations (2-1) to (2-3), the symbol I denotes electric current flowing through the antenna 39, and the symbol S denotes the area of the circular coil constructing the antenna 39. The symbol r denotes a distance between the antenna 39 and an arbitrary position (r=(x²+y²+z²)^{1/2}). Moreover, k=ω(∈μ)^{1/2} (where ∈ is a dielectric constant and μ is magnetic permeability), and the symbol j denotes an imaginary unit.

If a frequency of a magnetic field generated by the antenna 39 placed in the capsule endoscope 3 is high, and the capsule endoscope 3 is sufficiently apart from the receiving antenna 40(n) attached to the body surface of the subject 2, the radiation field component is the largest in the magnetic field (electromagnetic wave) reaching each of the receiving antennas 40(n). Therefore, the electrostatic field and induction field components are smaller than the radiation field component and accordingly, they can be ignored. Hence, equations (2-1) to (2-3) can be modified as in the following equations (3-1) to (3-3).

$$\begin{matrix}{H}_{r} = 0 & ( 3 \text{-} 1 ) \\ {H}_{θ} = \frac{IS}{4 π} ( - \frac{{k}^{2}}{r} ) \cdot {e}^{- j kr} \cdot sin θ & ( 3 \text{-} 2 ) \\ {E}_{ψ} = - \frac{j ω μ IS}{4 π} ( \frac{j k}{r} ) \cdot {e}^{- j kr} \cdot sin θ & ( 3 \text{-} 3 )\end{matrix}$$

Assuming that the receiving antennas 40(n) attached to the body surface of the subject 2 are electric-field detection antennas for detecting an electric field, an equation necessary for the detection is equation (3-3). The electric field component E_{ψ} given by equation (3-3) denotes a radiation electric field, and is considered to be the result by the AC theory. Therefore, an instantaneous value of the electric field component E_{ψ} is obtained by multiplying both sides of equation (3-3) by exp(jψt) as in the following equation (4), and extracting a real part.

$$\begin{matrix}{E}_{ψ} {e}^{j ω t} = - \frac{j ω μ IS}{4 π} \cdot \frac{j k}{r} \cdot {e}^{- j kr} \cdot sin θ \cdot {e}^{jω t} \\ = \frac{ω μ ISk}{4 π r} ( cos U + j sin U ) \cdot sin θ\end{matrix} \qquad ( 4 )$$

In equation (4), U=ψt−kr.

If the real part of equation (4) is extracted, an instantaneous value E′_{ψ} of the electric field is given by the following equation (5).

$${E}_{ψ}^{′} = \frac{ω μ ISk}{4 π r} cos U \cdot sin θ \qquad ( 5 )$$

Moreover, if equation (4) is converted from a polar coordinate system (r, θ, ψ) into an orthogonal coordinate system (X_{L}, Y_{L}, Z_{L}) as illustrated in FIG. 12, coordinate components (E_{Lx}, E_{Ly}, E_{Lz}) of the instantaneous value E′_{ψ} of the electric field are given by the following equations (6-1) to (6-3).

$$\begin{matrix}\begin{matrix}{E}_{Lx} = {E}_{ψ}^{′} \cdot sin ψ \\ = \frac{ω μ ISk}{4 π {r}^{2}} \cdot cos U \cdot ( - {y}_{L} )\end{matrix} & ( 6 \text{-} 1 ) \\ \begin{matrix}{E}_{Ly} = {E}_{ψ}^{′} \cdot cos ψ \\ = \frac{ω μ ISk}{4 π {r}^{2}} \cdot cos U \cdot {x}_{L}\end{matrix} & ( 6 \text{-} 2 ) \\ {E}_{Lz} = 0 & ( 6 \text{-} 3 )\end{matrix}$$

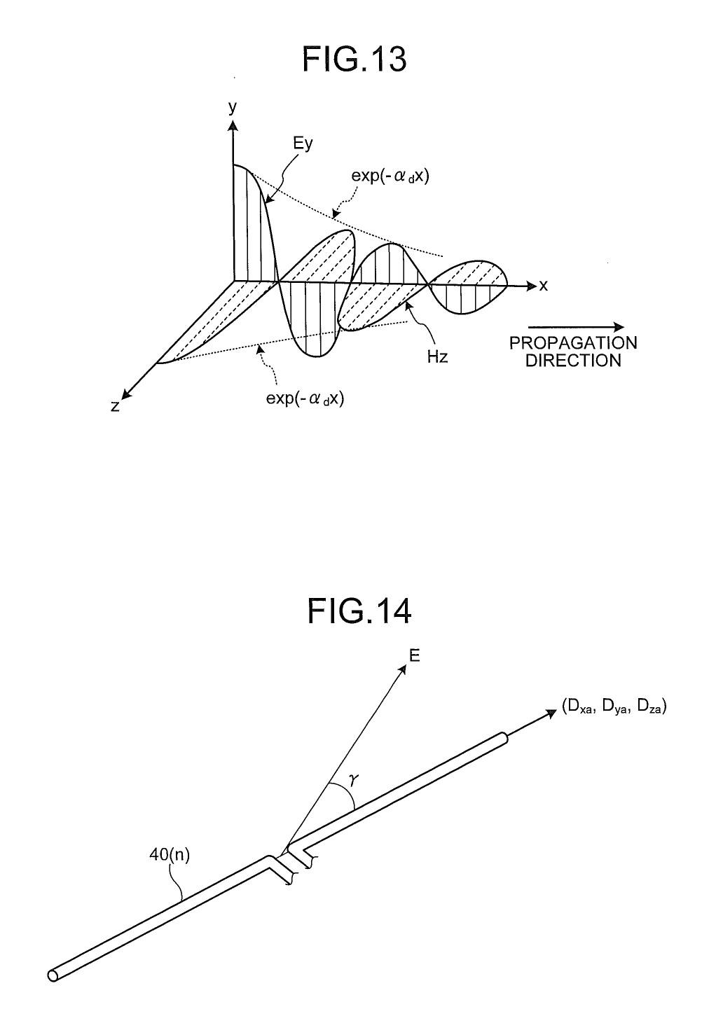

As illustrated in FIG. 13, if electromagnetic waves E_{y} and H_{z} propagate through a medium, the energy of the electromagnetic waves is absorbed in the medium in accordance with the characteristics of the medium such as electrical conductivity. For example, energy Aᵣ of the electromagnetic waves E_{y} and H_{z} propagating in the x direction is exponentially attenuated by a damping factor α_{d} as expressed by the following equations (7) and (8).

$$\begin{matrix}{A}_{r} = {e}^{- {α}_{d} x} & ( 7 ) \\ {α}_{d} = {( \frac{{ω}^{2} ɛ μ}{2} )}^{\frac{1}{2}} {\{ {( 1 + \frac{{k}^{2}}{{ω}^{2} {ɛ}^{2}} )}^{\frac{1}{2}} - 1 \}}^{\frac{1}{2}} & ( 8 )\end{matrix}$$

In equation (8), ∈=∈ₒ∈ᵣ (∈ₒ: dielectric constant of a vacuum, ∈r: dielectric constant of a medium), μ=μₒμᵣ (μₒ: magnetic permeability of a vacuum, μᵣ: magnetic permeability of a medium), ω is an angular frequency, k is electric conductivity.

Therefore, an instantaneous value E_{L} of an electric field when the characteristics in the living body are considered is given by the following equations (9-1) to (9-3).

$$\begin{matrix}\begin{matrix}{E}_{Lx} = {A}_{r} {E}_{ψ}^{′} sin ψ \\ = {e}^{- {α}_{d} r} \cdot \frac{ω μ ISk}{4 π {r}^{2}} \cdot cos U \cdot ( - {y}_{L} )\end{matrix} & ( 9 \text{-} 1 ) \\ \begin{matrix}{E}_{Ly} = {A}_{r} {E}_{ψ}^{′} cos ψ \\ = {e}^{- {α}_{d} r} \cdot \frac{ω μ ISk}{4 π {r}^{2}} \cdot cos U \cdot {x}_{L}\end{matrix} & ( 9 \text{-} 2 ) \\ {E}_{Lz} = 0 & ( 9 \text{-} 3 )\end{matrix}$$

Moreover, an equation that converts the position Q (x_{L}, y_{L}, z_{L}) in the coordinate system X_{L}Y_{L}Z_{L} relative to the antenna 39 of the capsule endoscope 3 into a coordinate system XYZ relative to the subject 2 is as in the following equation (10).

$$\begin{matrix}( \begin{matrix}{x}_{LP} \\ {y}_{LP} \\ {z}_{LP}\end{matrix} ) = {R}^{- 1} [ ( \begin{matrix}{x}_{WP} \\ {y}_{WP} \\ {z}_{WP}\end{matrix} ) - ( \begin{matrix}{x}_{WG} \\ {y}_{WG} \\ {z}_{WG}\end{matrix} ) ] \\ = ( \begin{matrix}{R}_{00} & {R}_{01} & {R}_{02} \\ {R}_{10} & {R}_{11} & {R}_{12} \\ {R}_{20} & {R}_{21} & {R}_{22}\end{matrix} ) [ ( \begin{matrix}{x}_{WP} \\ {y}_{WP} \\ {z}_{WP}\end{matrix} ) - ( \begin{matrix}{x}_{WG} \\ {y}_{WG} \\ {z}_{WG}\end{matrix} ) ]\end{matrix} \qquad ( 10 )$$

In equation (10), (x_{WP}, y_{WP}, z_{WP}) and (X_{WG}, y_{WG}, z_{WG}) represent the position Q and the position of the antenna 39 in the coordinate system X_{W}Y_{W}Z_{W}, respectively.

The matrix having, as components, R₀₀ to R₂₂ shown on the right side of equation (10) represents a rotation matrix of the coordinate system X_{W}Y_{W}Z_{W} and the coordinate system X_{L}Y_{L}Z_{L}, and is given by the following equation (11).

$$( \begin{matrix}{R}_{00} & {R}_{10} & {R}_{20} \\ {R}_{01} & {R}_{11} & {R}_{21} \\ {R}_{02} & {R}_{12} & {R}_{22}\end{matrix} ) = ( \begin{matrix}cos αcos β & - sin α & cos α sin β \\ sin α cos β & cos α & sin α sin β \\ - sin β & 0 & cos β\end{matrix} ) \qquad ( 11 )$$

In equation (11), α and β denote the rotation amounts in a polar coordinate system, respectively.

Consequently, an electric field E_{W} at the position Q (x_{WP}, y_{WP}, z_{WP}) in the coordinate system XYZ relative to the subject 2 is given by the following equation (2).

$$\begin{matrix}( \begin{matrix}{E}_{Wx} \\ {E}_{Wy} \\ {E}_{Wz}\end{matrix} ) = R ( \begin{matrix}{E}_{Lx} \\ {E}_{Ly} \\ {E}_{Lz}\end{matrix} ) \\ = ( \begin{matrix}{R}_{00} & {R}_{10} & {R}_{20} \\ {R}_{01} & {R}_{11} & {R}_{21} \\ {R}_{02} & {R}_{12} & {R}_{22}\end{matrix} ) ( \begin{matrix}{E}_{Lx} \\ {E}_{Ly} \\ {E}_{Lz}\end{matrix} )\end{matrix} \qquad ( 12 )$$

Equations (9-1) to (11) are substituted into equation (12) to obtain equation (13) that provides the electric field E_{W}.

$$( \begin{matrix}{E}_{Wx} \\ {E}_{Wy} \\ {E}_{Wz}\end{matrix} ) = \frac{{k}_{1}}{{r}^{2}} {e}^{- {α}_{d} r} ( \begin{matrix}0 & ( {z}_{WP} - {z}_{WG} ) & - ( {y}_{WP} - {y}_{WG} ) \\ ( {z}_{WP} - {z}_{WG} ) & 0 & ( {x}_{WP} - {x}_{WG} ) \\ ( {y}_{WP} - {y}_{WG} ) & - ( {x}_{WP} - {x}_{WG} ) & 0\end{matrix} ) ( \begin{matrix}{g}_{x} \\ {g}_{y} \\ {g}_{z}\end{matrix} ) \qquad ( 13 )$$

In equation (13), k₁ is a constant. Moreover, (gₓ, g_{y}, g_{z}) represents the orientation of the antenna 39 acquired in Step S14.

Therefore, the theoretical value Vtₙ(i) of the received strength detected when the receiving antenna 40(n) constructed of a dipole antenna receives the electric field E_{W} generated from the antenna 39 is given by the following equation (14).

$$\begin{matrix}{Vt}_{n} ( i ) = {k}_{2} {E}_{W} cos γ \\ = {k}_{2} ( {E}_{Wx} {D}_{xa} + {E}_{Wy} {D}_{ya} + {E}_{Wz} {D}_{za} )\end{matrix} \qquad ( 14 )$$

In equation (14), k₂ is a constant. Moreover, (Dₓₐ, D_{ya}, D_{za}) represents the orientation of the receiving antenna 40(n) in a coordinate system relative to the subject 2.

In the following Step S16, the position determination unit 623 calculates the sum M(i) of the absolute values of differences between the theoretical values Vtₙ(i) of the received strength of the receiving antennas 40(n) calculated in Step S15 and actual received strength (measured values) Vₙ at the antennas 40(n) by the following equation (15).

$$M ( i ) = {\sum }_{n = 1}^{N} \vert {Vt}_{n} ( i ) - {V}_{n} \vert \qquad ( 15 )$$

In the first embodiment, N=3.

The process of the loop A is repeated for all the lattice points Pi, and then in Step S17, the position determination unit 623 acquires the lattice point Pi having the minimum sum M(i) of the absolute values of the differences in the existing region T and the orientation Vc of the capsule endoscope 3 at that point in time.

Furthermore, in Step S18, the position determination unit 623 determines the position of the acquired lattice point Pi as the position of the capsule endoscope 3. At this point, the position determination unit 623 creates position information representing the determined position and stores the position information in the storage unit 64. Afterwards, the processing returns to the main routine.

In Step S4 (see FIG. 4) following Step S3, the locus calculator 63 executes a moving locus calculation process for calculating the moving locus of the capsule endoscope 3 in the subject 2 by connecting temporally neighboring positions of the capsule endoscope 3 based on the position information of the capsule endoscope 3 created by the position information estimating unit 62 and the position information of the capsule endoscope 3 hitherto stored in the storage unit 64.

The detected position of the capsule endoscope 3 may contain an error due to an error of arrangement of the receiving antennas 40(n), noise, and the like. For example, as illustrated in FIG. 15, a moving locus Lp linking the detected positions of the capsule endoscope 3 may deviate from an actual moving locus Lc of the capsule endoscope 3. However, the capsule endoscope 3 moves in the organs in the subject 2 and accordingly it is considered not to move greatly in a short time in reality.

Hence, the locus calculator 63 calculates the locus while performing a correction process such a median filtering process that obtains a median value from temporally neighboring coordinates (for example, three coordinates including the previous and subsequent ones). As a consequence, as illustrated in FIG. 16, the moving locust Lc that is closer to the actual moving locus Lp can be acquired. The moving locus Lc is one where the influence of a detected position A₁, which deviated greatly from the actual moving locus Lp due to an error of the arrangement of the receiving antennas 40(n), noise, and the like, has been reduced. Moreover, the correction process is not limited to the median filtering process, but may also use a moving average process for obtaining an average value of, for example, five coordinates including the two previous and following coordinates. The calculated moving locus is displayed on the display unit 66c together with an image where the inside of the subject 2 appears and is stored in the storage unit 64.

As described above, according to the first embodiment, the orientation of the capsule endoscope 3 is estimated, and the position of the capsule endoscope 3 is further narrowed down with the orientation from the existing region acquired based on the received strength at each of the receiving antennas 40(n). Accordingly, it becomes possible to perform position detection with higher accuracy than before.

Moreover, according to the first embodiment, a complicated calculation process becomes unnecessary and accordingly it becomes possible to enhance the speed of the position determination process and the moving locus calculation process.

Moreover, according to the first embodiment, the sheet-shaped receiving antenna unit 4 where the plurality of receiving antennas 40(n) is arranged is used. Accordingly, there is no need to adjust the arrangement of the receiving antennas 40(n) on every examination. Furthermore, the receiving antenna unit 4 where the arrangement of the receiving antennas 40(n) is determined in advance is used. Therefore, it is also possible to avoid a problem that the accuracy of the process of estimating the position of the capsule endoscope 3 is reduced with displacements of the receiving antennas 40(n).

In the first embodiment, when the position of the capsule endoscope 3 is determined, the sum of the absolute values of differences between theoretical values and measured values of received strength at the receiving antennas 40(n) is calculated. However, as long as a minimum value of an error between a theoretical value and a measured value can be found, another computation method may be used. For example, the sum of squared residuals of a theoretical value and a measured value may be calculated to detect the lattice point Pi having the minimum sum of squared residuals.

Moreover, in the first embodiment, a region where all spheres with three receiving antennas 40(n) as their centers and the distances rₙ corresponding respectively to the receiving antennas 40(n) as radii overlap with one another is acquired as the existing region of the capsule endoscope 3 (see FIG. 7). However, the capsule endoscope 3 is definitely located inside a region where at least two or more spheres overlap with one another. Hence, the existing region acquiring unit 621 may acquire, as the existing region of the capsule endoscope 3, a region where, for example, two spheres corresponding to two receiving antennas 40(n) of three receiving antennas 40(n) overlap with each other.

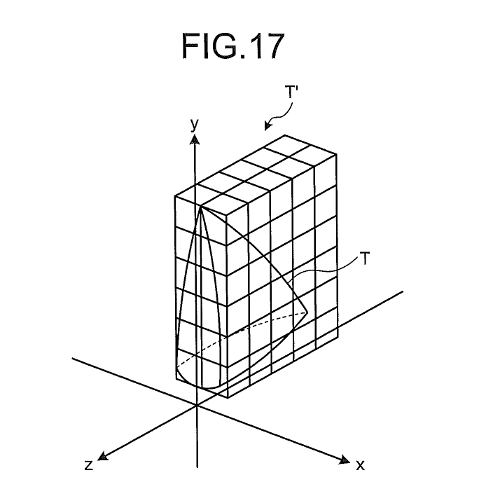

Moreover, in the first embodiment, the existing region T is acquired based on the receiving antennas 40(n) at every position detection timing. However, the capsule endoscope 3 is considered not to move greatly in a short time in reality so that the existing region T this time may be estimated based on the existing region T acquired last time. For example, as illustrated in FIG. 17, if the existing region T of the capsule endoscope 3 was acquired in the last position determination process, the process of the loop A (see FIG. 5) may be executed next time on the inside of a region T′ where an area where the capsule endoscope 3 can move is added to the existing region T.

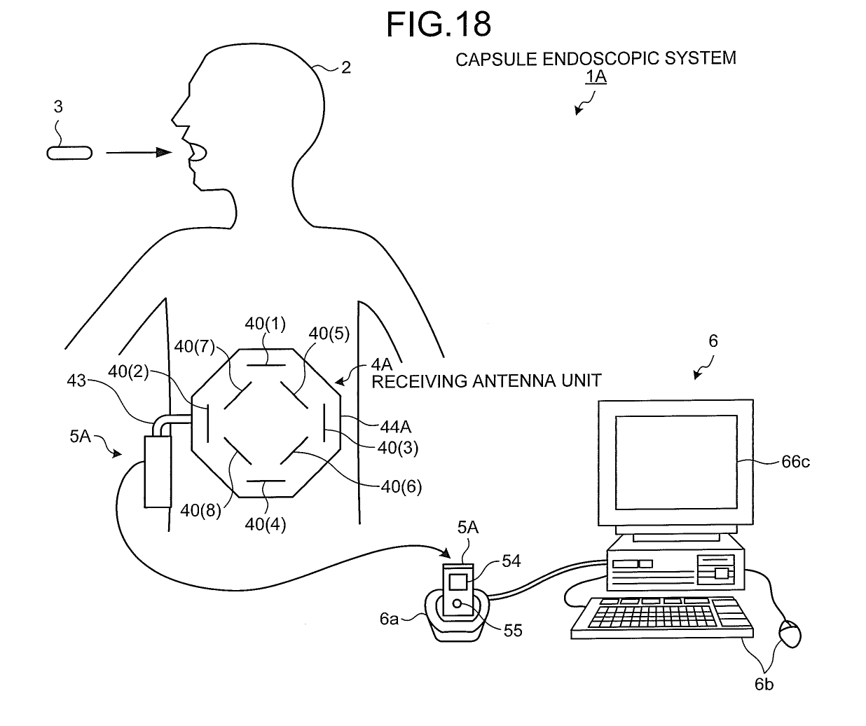

Moreover, in the first embodiment, the example where three receiving antennas 40(n) are provided to the receiving antenna unit 4 is described. However, there is no need to interpret the number of the receiving antennas limiting it to three. For example, a sheet 44A where eight receiving antennas 40(1) to 40(8) are arranged may be used as in a receiving antenna unit 4A connected to a receiving device 5A of a capsule endoscope system 1A illustrated in FIG. 18. Moreover, the arrangement of a plurality of the receiving antennas 40(n) may be optionally changed according to the purpose such as an examination or diagnosis.

### Second Embodiment

Next, a second embodiment of the present invention will be described.

In the first embodiment, the information processing device 6 including the position information estimating unit 62 and the locus calculator 63 estimates the position of the capsule endoscope 3 and calculates the locus. However, it may be configured such that an estimating unit that estimates position information and a locus calculator are provided on a receiving device side, and the position of the capsule endoscope 3 is estimated at a timing when the inside of the subject 2 is captured on the receiving device side. In this case, the receiving device functions as the position detection device. In the second embodiment, a configuration of the receiving device in this case will be described.

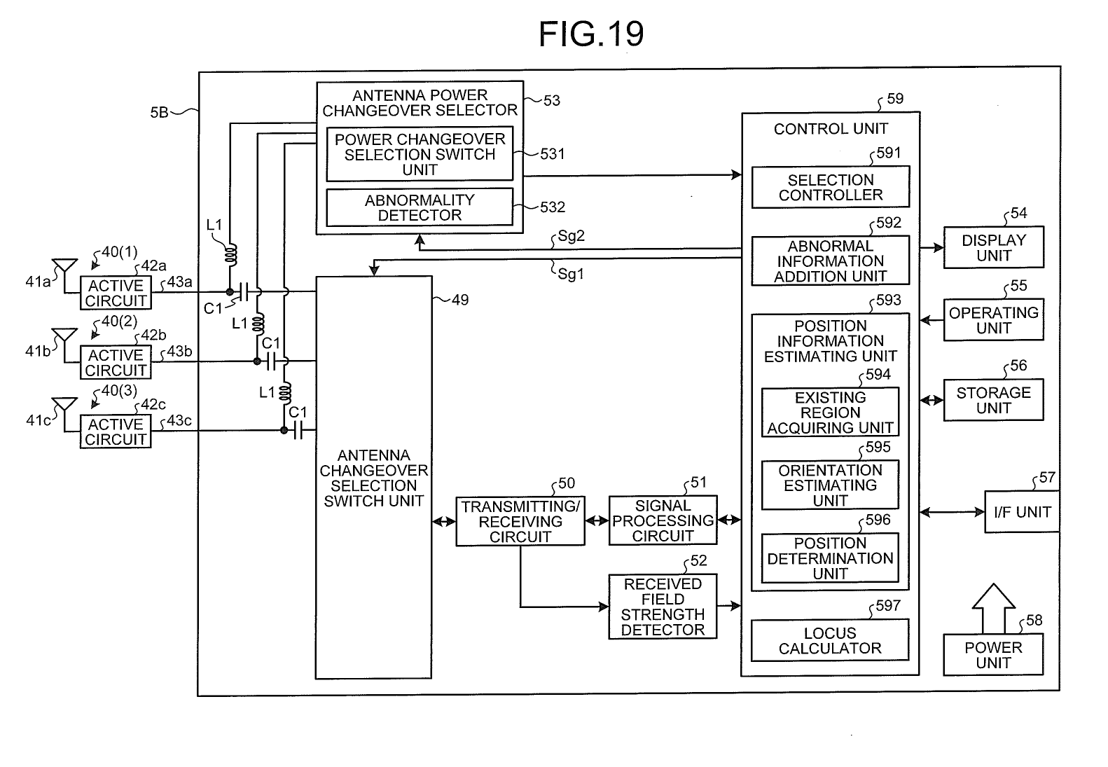

FIG. 19 is a block diagram illustrating a configuration example of a receiving device included in a capsule endoscope system according to the second embodiment. As illustrated in FIG. 19, a receiving device 5B includes the receiving antennas 40(n) (n=1 to 3), an antenna changeover selection switch unit 49 that alternatively switches between the receiving antennas 40(n), a transmitting/receiving circuit 50 that executes processes such as demodulation on a wireless signal received via the receiving antenna 40(n) selected by the antenna changeover selection switch unit 49, a signal processing circuit 51 that executes signal processing for extracting image data and the like from a wireless signal output from the transmitting/receiving circuit 50, a received field strength detector 52 that detects the received field strength based on the strength of the wireless signal output from the transmitting/receiving circuit 50, an antenna power changeover selector 53 that alternatively switches between the receiving antennas 40(1) to 40(3) and supplies electric power to any of the receiving antennas 40(1) to 40(3), the display unit 54 that displays an image corresponding to image data received from the capsule endoscope 3, the operating unit 55 used when an instruction operation on the receiving device 5B is input, a storage unit 56 that stores various pieces of information including image data received from the capsule endoscope 3, an I/F unit 57 that transmits and receives to and from an information processing device in a mutual direction via a cradle 6a, a power unit 58 that supplies electric power to the units of the receiving device 5B, and a control unit 59 that controls the operation of the receiving device 5B. Of them, the control unit 59 includes a position information estimating unit 593 having a similar function to that of the position information estimating unit 62 illustrated in FIG. 3, and a locus calculator 597 having a similar function to that of the locus calculator 63.

The receiving antenna 40(1) includes an antenna unit 41a, an active circuit 42a, and an antenna cable 43a. The antenna unit 41a is configured using, for example, a dipole antenna, and receives a wireless signal transmitted from the capsule endoscope 3. The active circuit 42a is connected to the antenna unit 41a, and performs such things as impedance matching of the antenna unit 41a, and amplification and attenuation of a received wireless signal. The antenna cable 43a is configured using a coaxial cable, is connected at one end to the active circuit 42a, and is electrically connected at the other end to the antenna changeover selection switch unit 49 and the antenna power changeover selector 53 of the receiving device 5B. The antenna cable 43a transmits a wireless signal received by the antenna unit 41a to the receiving device 5B and transmits electric power supplied from the receiving device 5B to the active circuit 42a. The receiving antennas 40(2) and 40(3) have a similar configuration to that of the receiving antenna 40(1). Accordingly, the description will be omitted.

The antenna changeover selection switch unit 49 is configured using a mechanical switch, a semiconductor switch, or the like. The receiving antenna 40(n) is electrically connected to the antenna changeover selection switch unit 49 via a capacitor C1. If a switching signal Sg1 that switches the receiving antenna 40(n) that receives a wireless signal is input from the control unit 59, the antenna changeover selection switch unit 49 selects the receiving antenna 40(n) instructed by the switching signal Sg1 and outputs a wireless signal received by the selected receiving antenna 40(n) to the transmitting/receiving circuit 50. The capacity of a capacitor connected to the receiving antenna 40(n) is equal to the capacity of the capacitor C1.

The transmitting/receiving circuit 50 performs predetermined processes such as a demodulation process and an amplification process on a wireless signal received by the receiving antenna 40(n) selected by the antenna changeover selection switch unit 49, and outputs the wireless signal to the signal processing circuit 51 and the received field strength detector 52.

The signal processing circuit 51 extracts image data from the wireless signal input from the transmitting/receiving circuit 50, performs predetermined processes such as various image processing and an A/D conversion process on the extracted image data, and outputs the image data to the control unit 59.

The received field strength detector 52 detects the received field strength in accordance with the strength of the wireless signal input from the transmitting/receiving circuit 50, and outputs to the control unit 59 a received signal strength indicator (RSSI) corresponding to the detected received field strength.

The receiving antenna 40(n) is electrically connected to the antenna power changeover selector 53 via a coil L1. The antenna power changeover selector 53 supplies electric power via the antenna cables 43 (43a to 43c) to the receiving antenna 40(n) selected by the antenna changeover selection switch unit 49. The electrical characteristic of the coil connected to the receiving antenna 40(n) is equal to that of the coil L1.

The antenna power changeover selector 53 includes a power changeover selection switch unit 531 and an abnormality detector 532. The power changeover selection switch unit 531 is configured using a mechanical switch, a semiconductor switch, or the like. If a selection signal Sg2 for selecting the receiving antenna 40(n) to which power is supplied is input from the control unit 59, the power changeover selection switch unit 531 selects the receiving antenna 40(n) instructed by the selection signal Sg2, and power is supplied only to the selected receiving antenna 40(n).

If an abnormality is occurring at the receiving antenna 40(n) being a power supply destination, the abnormality detector 532 outputs to the control unit 59 an abnormal signal indicating the occurrence of the abnormality at the receiving antenna 40(n) being the power supply destination.

The display unit 54 is configured using a display panel including liquid crystals, organic EL (Electro Luminescence), or the like. The display unit 54 displays various pieces of information such as an image corresponding to image data captured by the capsule endoscope 3, the operating state of the receiving device 5B, patient information of the subject 2, and an examination date and time.

The operating unit 55 is used when instruction signals to perform such things as changing the image capture cycle of the capsule endoscope 3 are input. If the instruction signal is input via the operating unit 55, the signal processing circuit 51 transmits the instruction signal to the transmitting/receiving circuit 50. The transmitting/receiving circuit 50 modulates the instruction signal and transmits the instruction signal from the receiving antennas 40(1) to 40(3). The signals transmitted from the receiving antennas 40(1) to 40(3) are received by the antenna 39 of the capsule endoscope 3 (see FIG. 2), and demodulated by the transmitting/receiving circuit 37. The circuit board 36 performs an operation such as changing the image capture cycle in response to the instruction signal.

The storage unit 56 is configured using a semiconductor memory such as a flash memory or a RAM (Random Access Memory) provided fixedly in the receiving device 5B. Moreover, the storage unit 56 stores image data captured by the capsule endoscope 3 and various pieces of information associated with the image data, for example, estimated position information of the capsule endoscope 3, received field strength information, and identification information for identifying the receiving antenna that has received a wireless signal. Furthermore, the storage unit 56 stores various programs to be executed by the receiving device 5B, and the like. The storage unit 56 may be provided with a function as a recording medium interface that stores information in an external recording medium such as a memory card and reads the information stored in the recording medium.

The I/F unit 57 has a function as a communication interface, and transmits and receives to and from the information processing device 6 in a mutual direction via the cradle 6a.

The power unit 58 is configured using a battery detachable from the receiving device 5B and a switch unit that switches between an on state and an off state. The power unit 58 supplies driving power necessary for the configuration units of the receiving device 5B in the on state, and stops the driving power supplied to the configuration units of the receiving device 5B in the off state.

The control unit 59 is configured using a CPU (Central Processing Unit), and the like. The control unit 59 reads a program from the storage unit 56 and executes the program, and performs such things as instructing the units included in the receiving device 5B and transferring data to the units to perform centralized control of the operation of the receiving device 5B. The control unit 59 includes a selection controller 591, an abnormal information addition unit 592, a position information estimating unit 593, and a locus calculator 597.

The selection controller 591 executes control to select one receiving antenna 40(n) that receives a wireless signal transmitted from the capsule endoscope 3 and supply power only to the selected receiving antenna 40(n). Specifically, the selection controller 591 selects one receiving antenna 40(n) that is allowed to receive a wireless signal including an image signal transmitted from the capsule endoscope 3 at an image signal receiving timing, at an antenna selecting timing based on the field received strength of the receiving antennas 40(n) detected by the received field strength detector 52. Moreover, the selection controller 591 executes control to supply power only to the selected receiving antenna 40(n) at the image signal receiving timing. In order to sequentially select the receiving antenna 40(n) that is allowed to receive a wireless signal including an image signal from the plurality of receiving antennas 40(n), for example, at intervals of 100 msec as the antennal selecting timing, the selection controller 591 causes the received field strength detector 52 to detect the received field strength of the receiving antennas 40(n), and then drives the antenna changeover selection switch unit 49 to supply power only to the selected one receiving antenna 40(n).

If the abnormality detector 532 detects an abnormality in any of the three receiving antennas 40(n), the abnormal information addition unit 592 adds abnormal information indicating the occurrence of the abnormality in any of the receiving antennas 40(n) to a wireless signal received by the receiving antenna 40(n). Specifically, the abnormal information addition unit 592 adds abnormal information (flag) to image data generated by the signal processing circuit 51 performing signal processing on the wireless signal received by the receiving antenna 40(n), outputs the image data, and stores the image data in the storage unit 56.

The position information estimating unit 593 includes an existing region acquiring unit 594 having a similar function to that of the existing region acquiring unit 621 illustrated in FIG. 3, an orientation estimating unit 595 having a similar function to that of the orientation estimating unit 622, and a position determination unit 596 having a similar function to that of the position determination unit 623.

As described above, according to the second embodiment, the position information estimating unit 593 is provided in the receiving device 5B. Accordingly, it becomes possible to detect the position of the capsule endoscope 3 in the subject 2 with high accuracy and in real time. Moreover, according to the second embodiment, it is possible to cut a calculation amount in the position information estimating unit 593. Accordingly, it becomes possible to detect the position of the capsule endoscope 3 without significantly increasing a load on the receiving device 5B.

According to the above-described first and second embodiments, the orientation of the capsule endoscope is estimated, and the position of the capsule endoscope is further narrowed down with the orientation from a region acquired based on the received strength at each receiving antenna and accordingly it becomes possible to detect the position of the capsule endoscope with higher accuracy than before while cutting the calculation amount.

The above-described embodiments and their modifications are just examples for carrying out the present invention, and the present invention is not limited to them. Moreover, various inventions can be derived from combinations of a plurality of components disclosed in the embodiments and their modifications of the present invention. Various modifications can be made according to specifications, and it is obvious from the above disclosures that other various embodiments may be made within the scope of the present invention.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A position detection device for detecting a position of a capsule endoscope in a subject, the capsule endoscope being introduced into the subject and moving in the subject, based on received strength of signals transmitted from the capsule endoscope at a plurality of receiving antennas, the position detection device comprising:
a region acquiring unit that obtains distances between the receiving antennas and the capsule endoscope based on the received strength of the signals received by the plurality of receiving antennas, and acquires a region where at least two spheres of a plurality of spheres with the receiving antennas as centers and the distances corresponding respectively to the receiving antennas as radii overlap with one another;
an orientation estimating unit that estimates orientations of the capsule endoscope at a plurality of points in the region based on positional relationships between the points and the receiving antennas, and the received strength of the signals received by the receiving antennas; and
a position determination unit that determines the position of the capsule endoscope in the subject based on the positions of the points and the orientations of the capsule endoscope,
wherein the position determination unit calculates theoretical values of the received strength of the signals received by the plurality of receiving antennas based on the positions of the points and the orientations of the capsule endoscope in a case of assuming that the capsule endoscope exists at the points, as well as acquiring measured values of the received strength at the plurality of receiving antennas, and detects a point having a minimum error between the theoretical value and the measured value as the position of the capsule endoscope out of the plurality of points.

2. The position detection device according to claim 1, wherein the orientation estimating unit acquires a candidate region of a point at which a vector representing the orientation of the capsule endoscope passes based on a direction of the receiving antenna, a direction from the point to the receiving antenna, and the received strength at the receiving antenna, and determines, as the point at which the vector passes, a position where the candidate regions of the point at which the vector passes, the candidate regions being acquired respectively for the plurality of receiving antennas, overlap with one another.

3. The position detection device according to claim 1, wherein the error is a sum of absolute values of differences or a sum of squared residuals between the theoretical values and the measured values at the receiving antennas.

4. The position detection device according to claim 1, further comprising a locus calculator that calculates a moving locus of the capsule endoscope based on the position of the capsule endoscope determined by the position determination unit.

5. The position detection device according to claim 4, further comprising an image display unit that displays the moving locus of the capsule endoscope in the subject, the moving locus being calculated by the locus calculator.

6. A capsule endoscope system comprising:
a capsule endoscope that is introduced into a subject and moves in the subject to acquire image information inside the subject; and
a position detection device that detects a position of the capsule endoscope in the subject based on received strength of signals transmitted from the capsule endoscope at a plurality of receiving antennas, the position detection device including a region acquiring unit for obtaining distances between the receiving antennas and the capsule endoscope based on the received strength of the signals received by the plurality of receiving antennas, and acquiring a region where at least two spheres of a plurality of spheres with the receiving antennas as centers and the distances corresponding respectively to the receiving antennas as radii overlap with one another, an orientation estimating unit for estimating orientations of the capsule endoscope at a plurality of points in the region based on positional relationships between the points and the receiving antennas, and the received strength of the signals received at the receiving antennas, and a position determination unit for determining the position of the capsule endoscope in the subject based on the positions of the points and the orientations of the capsule endoscope,
wherein the position determination unit calculates theoretical values of the received strength of the signals received by the plurality of receiving antennas based on the positions of the points and the orientations of the capsule endoscope in a case of assuming that the capsule endoscope exists at the points, as well as acquiring measured values of the received strength at the plurality of receiving antennas, and detects a point having a minimum error between the theoretical value and the measured value as the position of the capsule endoscope out of the plurality of points.

7. The capsule endoscope system according to claim 6, wherein the receiving antennas are antennas that use only main polarization.

8. The capsule endoscope system according to claim 7, wherein the receiving antennas are dipole antennas.

9. A capsule endoscope system comprising:
a capsule endoscope that is introduced into a subject and moves in the subject to acquire image information inside the subject;
a receiving device including a plurality of antennas for receiving a signal including the image information transmitted from the capsule endoscope, a region acquiring unit for obtaining distances between the receiving antennas and the capsule endoscope based on received strength of the signals received by the plurality of receiving antennas, and acquiring a region where at least two spheres of a plurality of spheres with the receiving antennas as centers and the distances corresponding respectively to the receiving antennas as radii overlap with one another, an orientation estimating unit for estimating orientations of the capsule endoscope at a plurality of points in the region based on positional relationships between the points and the receiving antennas, and the received strength of the signals received by the receiving antennas, and a position determination unit for determining a position of the capsule endoscope in the subject based on the positions of the points and the orientations of the endoscope; and
an image display unit that acquires the image information and position information indicating the position of the capsule endoscope corresponding to the image information from the receiving device and displays an image corresponding to the image information and the position of the capsule endoscope,
wherein the position determination unit calculates theoretical values of the received strength of the signals received by the plurality of receiving antennas based on the positions of the points and the orientations of the capsule endoscope in a case of assuming that the capsule endoscope exists at the points, as well as acquiring measured values of the received strength at the plurality of receiving antennas, and detects a point having a minimum error between the theoretical value and the measured value as the position of the capsule endoscope out of the plurality of points.

10. A non-transitory computer readable recording medium where an executable program is stored, the program instructing a processor to execute the following:
obtaining distances between receiving antennas and a capsule endoscope that is introduced into a subject and moves in the subject, based on received strength at the receiving antennas upon the receiving antennas receiving signals transmitted from the capsule endoscope, and acquiring a region where at least two spheres of a plurality of spheres with the receiving antennas as centers and the distances corresponding respectively to the receiving antennas as radii overlap with one another;
estimating orientations of the capsule endoscope at a plurality of points in the region based on positional relationships between the points and the receiving antennas, and the received strength of the signals received by the receiving antennas;
determining a position of the capsule endoscope in the subject based on the positions of the points and the orientations of the capsule endoscope; and
upon determination of the position, calculating theoretical values of the received strength of the signals received by the plurality of receiving antennas based on the positions of the points and the orientations of the capsule endoscope in a case of assuming that the capsule endoscope exists at the points, as well as acquiring measured values of the received strength at the plurality of receiving antennas, and detecting a point having a minimum error between the theoretical value and the measured value as the position of the capsule endoscope out of the plurality of points.
